(19) United States
(12) Patent Application Publication
(10) Pub. No.: US 2017/0035461 A1
(43) Pub. Date: Feb. 9, 2017

(54) REVERSIBLE TATTOO COLORANT AND METHOD

(71) Applicant: Jeffery Deal, Charleston, SC (US)

(72) Inventor: Jeffery Deal, Charleston, SC (US)

(21) Appl. No.: 15/227,569

(22) Filed: Aug. 3, 2016

Related U.S. Application Data
(60) Provisional application No. 62/200,171, filed on Aug. 3, 2015.

(51) Int. Cl. A61B 17/52; A61B 17/50

(52) U.S. Cl. CPC A61B 17/52; A61B 17/50

(57) ABSTRACT

A tattoo ink or colorant has magnetic properties. The ink is delivered to the proper depth of the skin. The colorant remains in place, forming the desired visible design. When removal of the tattoo is desired, a magnet is topically applied to draw the ink or colorant to move the nanoparticles to the epidermis. The nanoparticles are then shed from the epidermis along with squamous cell debris.

Applicant claims the benefit of Provisional Application Ser. No. 62/200,171 filed Aug. 3, 2015.

### FIELD OF THE INVENTION

This invention relates to tattoos and a colorant system for forming tattoos that may be removed from the skin using a magnet.

### BACKGROUND OF THE INVENTION

A recent study from the British Journal of Dermatology reports that one-third of people with tattoos regret getting them. This supports the 2012 Harris Poll finding that 21% of the US population has tattoos and at least 14% of these regret getting a tattoo. Also, according to the American Society for Aesthetic Plastic Surgery (ASAPS), the number of people undergoing laser tattoo removal increased by 43% from 2011 to 2012.

While several removable tattoos exist on the market, most are either completely temporary and rely upon washable pigments or are formed of particles that degrade with exposure to certain lasers. The latter process does not remove the particles, but breaks the particles for absorption by the body. Accordingly, laser “removal” has been associated with health concerns.

Other methods of tattoo removal include dermabrasion, cryobrasion, chemabrasion, and complete excision. These methods either destroyed the epidermis and then the tattoo itself, or cut out the tattoo entirely. These methods tend to produce a significant degree of scar tissue.

### SUMMARY OF THE INVENTION

A tattoo ink or colorant has magnetic properties. The ink is delivered to the proper depth of the skin. The colorant remains in place, forming the desired visible design. When removal of the tattoo is desired, a magnet is topically applied to draw the ink or colorant to move the nanoparticles to the epidermis. The nanoparticles are then shed from the epidermis along with squamous cell debris.

### BRIEF DRAWING DESCRIPTION

FIG. 1 is a sectioned view of the dermis showing ink comprising paramagnetic material newly deposited by a needle in an upper surface of the dermis.

FIG. 2 is the view of FIG. 1 showing an initial location of ink after complete deposit of the ink comprising paramagnetic material at an injection site.

FIG. 3 is the view of FIG. 2 showing positioning of ink comprising paramagnetic material after an additional passage of time.

FIG. 4 is the view of FIG. 3 showing positioning of ink comprising paramagnetic material after a further passage of time.

FIG. 5 shows ink comprising paramagnetic material being withdrawn into the upper surface of the dermis by magnetism.

### DESCRIPTION OF PREFERRED EMBODIMENTS

A colorant for tattoos comprises chemically stable and inert (when applied to the skin) nanoparticles comprising sufficient ferromagnetic, supermagnetic, or paramagnetic properties to be attracted to a magnet that is topically applied. One nanoparticle formulation according to the invention comprises a supermagnetic iron oxide core iron (Ferrous) core surrounded or encapsulated by an inert shell. Shells may be produced in multiple colors for application in a desired design by a tattoo artist. Without being bound by theory, it is believed that nanoparticles, as described, may be constructed for application by known tattoo delivery systems and devices. Upon delivery to the proper depth of the skin, the colorant remains in place, forming the desired visible design.

In a preferred embodiment, the ink comprises paramagnetic material. Paramagnetic materials include gadolinium, magnesium, molybdenum, lithium, and tantalum. Any paramagnetic material delivered into the skin must be in a form having sufficiently low levels of toxicity. Other potential paramagnetic materials applicable to this use are superparamagnetic iron platinum, various other iron oxide compounds, and additional rare earth metals.

In a particular preferred embodiment, the color or ink formulation comprises chelated gadolinium, such as C₁₆H₂₅GdN₄O₈, with or without additional coloring particles. Chelated gadolinium is approved by the Federal Food and Drug Administration for injection into humans, and the potential for oxidation is reduced.

Pigment particles in this application are preferred to be slightly larger than nanoparticle size (greater than 100 nanometers) to reduce biological activity and enhance retention in the dermis. Once placed, the ink remains in place like conventional tattoos until magnetic removal is performed.

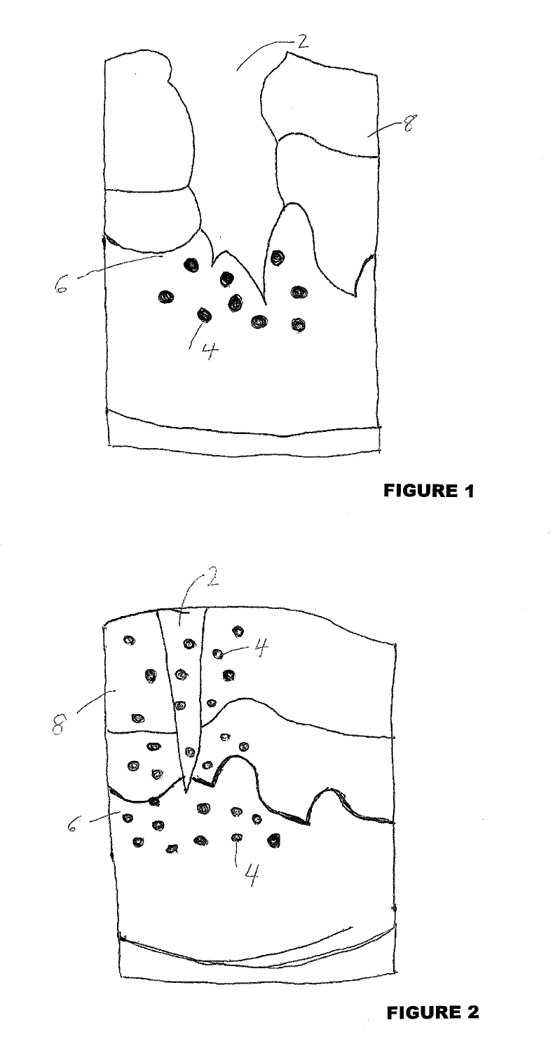

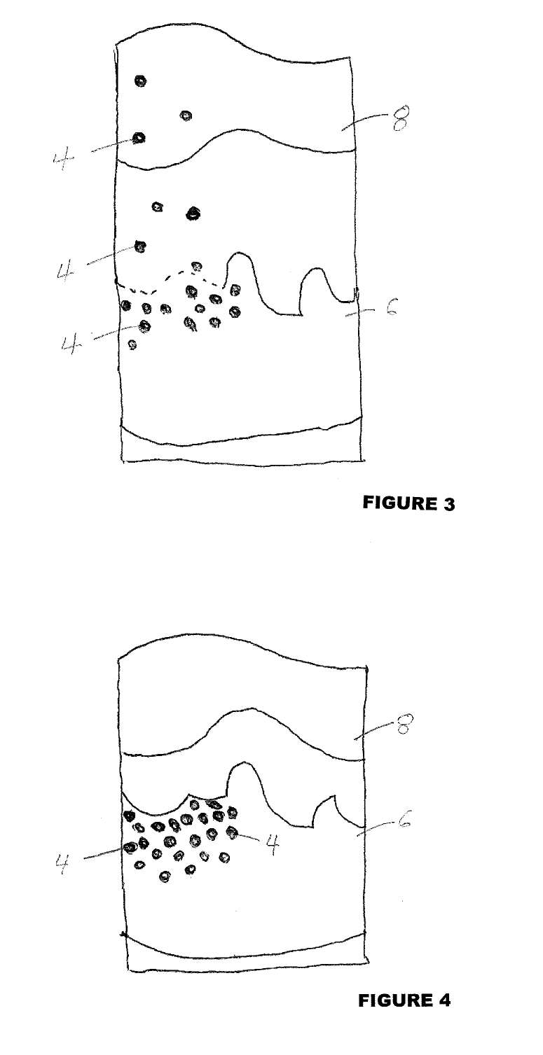

FIG. 1 shows a tattoo needle injection site 2. Ink 4 comprising paramagnetic material is deposited in the upper surface of the dermis 6. FIG. 2 shows ink location soon after the tattoo is received. The injection site has begun to heal. FIG. 3 shows migration or ejection of the ink from the epidermis 8 after an additional period of time, and the injection site is substantially healed. FIG. 4 shows the ink after it has finally been ejected from the epidermis into the upper dermis 6, which may be a period of 2-3 months from injection of the ink.

During the ink migration shown in FIGS. 1 through 4, phagocytes flock to the site of the tattoo, eject the ink from the epidermis, and engulf the ink injected into the dermis. The ink in the dermis is too far down to easily eject, and is engulfed in a granulation layer, which turns into connective tissue. Eventually, the pigment is trapped in fibroblasts, in a discrete layer created between the upper dermis and the epidermis. FIG. 4. Fibroblasts do not regenerate like regular cells, and therefore, they hold the ink in one place.

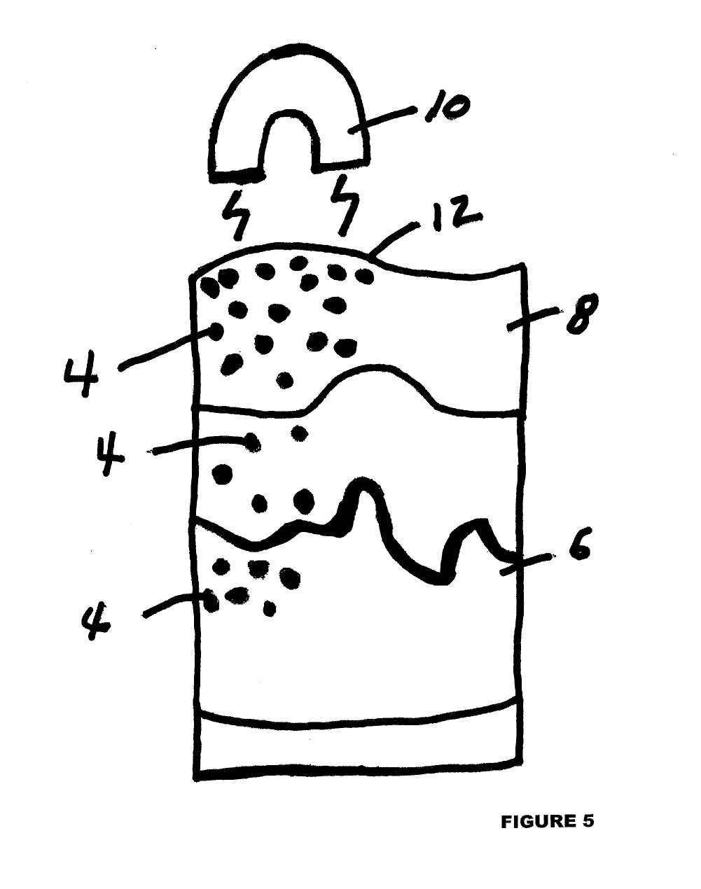

When removal is desired, a magnet 10 is applied over the surface 12 of the skin and over the tattoo for the required time to move the ink particles 4 comprising the paramagnetic material to the epidermis. FIG. 5. The ink is shed from the epidermis over time along with squamous cell debris as the epidermis normally sheds in a healthy person.

In one embodiment, a rigid or flexible magnet is worn over the tattoo and in contact with the epidermis for the required time to move the nanoparticles to the epidermis. Another variation of tattoo removal according to the invention uses magnets, which may be electromagnets, employed in multiple applications and at spaced apart time intervals to the skin to accomplish removal. The magnet may be a flexible magnet that conforms to contours of the skin.

In addition to body art tattoos, the colorant may be used to form reversible/changeable cosmetics, bar code identifications for animals, radiation therapy planning, tumor monitoring, and other applications where the formation of a tattoo on the skin is useful, but not desired for an indefinite period.

The invention provides a material and a process for removing ink or colorants from the body rather than absorption into the reticuloendothelial system. Removal of the tattoo may be accomplished with low-tech magnetic devices rather than regulated laser devices, thereby lowering the cost of tattoo removal.

What is claimed:

1. A process of applying and removing a tattoo, comprising the steps of:
preparing an ink, the ink comprising a colorant and a paramagnetic material;
injecting the ink into upper dermis;
after a passage of time, positioning a magnet over epidermis that is adjacent to upper dermis at the location of the ink;
withdrawing the ink into the epidermis by magnetic attraction of the ink to the magnet; and
allowing the epidermis and the ink in the epidermis to be shed.

2. The process of applying and removing a tattoo of claim 1, wherein the paramagnetic material is chelated gadolinium.

3. The process of applying and removing a tattoo of claim 1, wherein the paramagnetic material is larger than a nanoparticle.

4. The process of applying and removing a tattoo of claim 1, wherein the magnet is an electromagnet.

5. The process of applying and removing a tattoo of claim 1, wherein the magnet is applied in multiple applications at spaced apart time intervals.

6. The process of applying and removing a tattoo of claim 1, wherein the magnet is a flexible magnet that conforms to outer contours of the skin and is topically applied.

7. The process of applying and removing a tattoo of claim 1, wherein the ink is injected into the upper dermis by a tattoo needle.

8. The process of applying and removing a tattoo of claim 1, wherein the ink is withdrawn by the magnet worn over the skin and above the ink for sufficient time to draw the ink into the epidermis.

9. The process of applying and removing a tattoo of claim 1, wherein the ink is withdrawn by the magnet worn over the skin and above the ink for sufficient time to draw the ink into the epidermis and the magnet is a flexible magnet that conforms to outer contours of the skin and is topically applied.
